(19) United States
(12) Patent Application Publication
(10) Pub. No.: US 2006/0067990 A1
(43) Pub. Date: Mar. 30, 2006

(54) Absorbent articles for inhibiting the production of exoproteins

(75) Inventors: Rae Ellen Syverson, Fond du Lac, WI (US); Kristin A. Goerg-Wood, Sherwood, WI (US); Daniel J. Devine, Menasha, WI (US)

Correspondence Address: SENNIGER POWERS, ONE METROPOLITAN SQUARE, 16TH FLOOR, ST LOUIS, MO, 63102, US

(73) Assignee: Kimberly-Clark Worldwide, Inc.

(21) Appl. No.: 10/955,821

(22) Filed: Sep. 30, 2004

(51) Int. Cl. A61K 9/70

(52) U.S. Cl. 424/443

(57) ABSTRACT

Absorbent articles for inhibiting the production of exoproteins from Gram positive bacteria are disclosed. The absorbent articles include an effective amount of a precursor compound having the general formula: 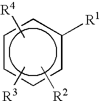
wherein R¹ is selected from the group consisting of 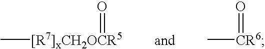
R⁷ is —OCH₂—; X is 0 or 1; R⁵ is a substituted or unsubstituted aromatic ring or a monovalent saturated or unsaturated, substituted or unsubstituted, branched or straight chain hydrocarbyl moiety that may or may not be substituted with hetero atoms; R⁶ is selected from the group consisting of an amino acid, a methyl ester of an amino acid, and an ethyl ester of an amino acid; R², R³, and R⁴ are independently selected from the group consisting of H, OH, COOH.

### BACKGROUND OF INVENTION

The present invention relates to the inhibition of exoprotein production in and around a woman's vagina in association with an absorbent article. More particularly, the present invention relates to the incorporation of a precursor compound into or onto an absorbent article such that upon use, the precursor compound can be hydrolyzed by enzymatic activity in and around the vagina to produce an active species that reduces the production of exoproteins from bacteria.

Disposable absorbent articles, such as vaginal tampons, for the absorption of vaginal exudates are widely used. These disposable articles typically have a compressed mass of absorbent formed into the desired shape, which is typically dictated by the intended consumer use. In the area of a vaginal tampon, the device is intended to be inserted into the vagina for absorption of the body fluids generally discharged during a woman's menstrual period.

There exists in the female body a complex process that maintains the vagina and physiologically related areas in a healthy state. In a female between the age of menarche and menopause, the normal vagina provides an ecosystem for a variety of microorganisms. Bacteria are the predominant type of microorganism present in the vagina, and most women harbor about 10⁹ bacteria per gram of vaginal fluid. The bacterial flora of the vagina is comprised of both aerobic and anaerobic bacteria. The more commonly isolated bacteria are Lactobacillus species, Corynebacteria species, Gardnerella vaginalis, Staphylococcus species, Peptococcus species, aerobic and anaerobic Streptococcus species, and Bacteroides species. Other microorganisms that have been isolated from the vagina on occasion include yeast (Candida albicans), protozoa (Trichomonas vaginalis), mycoplasma (Mycoplasma hominis), chlamydia (Chlamydia trachomatis), and viruses (Herpes simplex). These latter organisms are generally associated with vaginitis or venereal disease, although they may be present in low numbers without causing symptoms.

Physiological, social, and idiosyncratic factors affect the quantity and species of bacteria present in the vagina. Physiological factors include age, day of the menstrual cycle, and pregnancy. For example, microorganisms present in the vagina throughout the menstrual cycle can include lactobacilli, corynebacterium, ureaplasma, and mycoplasma. The number of microorganisms and the types of microorganisms are unique to an individual. Social and idiosyncratic factors include method of birth control, sexual practices, systemic disease (e.g., diabetes), and medications.

Bacterial proteins and metabolic products produced in the vagina can affect other microorganisms and the human host. For example, the vagina between menstrual periods is mildly acidic having a pH ranging from about 3.8 to about 4.5. This pH range is generally considered the most favorable condition for the maintenance of normal flora. At that pH, the vagina normally harbors the numerous species of microorganisms in a balanced ecology. These microorganisms play a beneficial role in providing protection and resistance to infection and make the vagina inhospitable to some species of bacteria such as Staphylococcus aureus (S. aureus). The low pH is a consequence of the growth of lactobacilli and their production of acidic products. Microorganisms in the vagina can also produce antimicrobial compounds such as hydrogen peroxide and bactericides directed at other bacterial species. One example is the lactocins, bacteriocin-like products of lactobacilli directed against other species of lactobacilli.

Some microbial products produced in the vagina may negatively affect the human host. For example, S. aureus can produce and excrete into its environment a variety of exoproteins including enterotoxins, Toxic Shock Syndrome Toxin-1 (TSST-1), and enzymes such as esterase and amidase. When absorbed into the bloodstream of the host, TSST-1 may lead to the development of Toxic Shock Syndrome (TSS) in non-immune humans.

S. aureus is found in the vagina of approximately 16% of healthy women of menstrual age. Not all strains of S. aureus can produce TSST-1. Approximately 25% of these women will harbor TSST-1 producing S. aureus. TSST-1 and some of the staphylococcal enterotoxins have been identified as causing TSS in humans.

Symptoms of TSS generally include fever, diarrhea, vomiting and a rash followed by a rapid drop in blood pressure. Multiple organ failure occurs in approximately 6% of those who develop the disease. S. aureus does not initiate TSS as a result of the invasion of the microorganism into the vaginal cavity. As S. aureus grows and multiplies, it can produce TSST-1. Only after entering the bloodstream does TSST-1 act systemically and produce the symptoms attributed to TSS.

Menstrual fluid has a pH of about 7.3. During menses, the pH of the vagina moves toward neutral and can become slightly alkaline. This change permits microorganisms whose growth is inhibited by an acidic environment to proliferate. For example, S. aureus is more frequently isolated from vaginal swabs during menstruation than from swabs collected between menstrual periods.

When S. aureus is present in an area of the human body that harbors a normal microbial population such as the vagina, it may be difficult to eradicate the S. aureus bacterium without harming members of the normal microbial flora required for a healthy ecosystem. Typically, antibiotics that kill S. aureus are not an option for use in products inserted into the vagina because of their effect on the normal vaginal microbial flora. An alternative to complete eradication is technology designed to prevent or substantially reduce the bacterium's ability to produce toxins.

There have been numerous attempts to reduce or eliminate pathogenic microorganisms and menstrually occurring TSS by incorporating one or more biostatic, biocidal, and/or detoxifying compounds into vaginal products. For example, L-ascorbic acid has been applied to a menstrual tampon to detoxify toxin found in the vagina. Others have incorporated monoesters and diesters of polyhydric aliphatic alcohols, such as glycerol monolaurate, as biocidal compounds (see, e.g., U.S. Pat. No. 5,679,369). Still others have introduced other non-ionic surfactants, such as alkyl ethers, alkyl amines, and alkyl amides as detoxifying compounds (see, e.g., U.S. Pat. Nos. 5,685,872, 5,618,554, and 5,612,045).

One significant problem associated with some of the above previous attempts as well as others is that the compounds used may be highly volatile during incorporation into absorbent articles and during further manufacturing processes. Specifically, it has been discovered that compounds, such as some aromatics, terpenes, and isoprenoids, are volatilized completely out of an absorbent product during high temperature manufacturing steps. Also, some compounds may have volatility issues during storage prior to use by the consumer.

As such, there continues to be a need for absorbent products comprising compounds that will effectively inhibit the production of exoproteins, such as TSST-1, from Gram positive bacteria without being substantially harmful to the natural flora found in the vaginal area. Additionally, these compounds need to maintain activity even in the presence of the enzymes lipase, esterase, and amidase, which can have adverse effects on potency and which may also be present in the vagina. It is desirable that the compounds have low volatility and remain in the product throughout manufacturing, storage, and transportation in order to deliver an effective inhibitor to the consumer.

### SUMMARY OF THE INVENTION

The present invention is directed to absorbent articles that inhibit the production of exoprotein from Gram positive bacteria. In one specific embodiment, the present invention is directed to a vaginal tampon incorporating one or more precursor compounds that are formed by linking one or more aromatic compounds to one or more secondary compounds via an ester or amide bond. Once introduced into the vagina, these precursor compounds can be hydrolyzed by enzymes produced by the vaginal flora resulting in an active species that can inhibit the production of exoprotein from Gram positive bacteria without substantially affecting the flora present in the vagina. In some embodiments, the precursor compound itself may also inhibit the production of exoprotein from Gram positive bacteria.

Therefore, the present invention is directed to an absorbent article for inhibiting the production of exoproteins from Gram positive bacteria. The absorbent article is suitable for insertion into the vagina and comprises an absorbent structure and an effective amount of a precursor compound having the general formula: 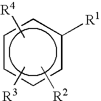
wherein R¹ is 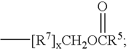
R⁷ is —OCH₂—; X is 0 or 1; R⁵ is a substituted or unsubstituted aromatic ring or a monovalent saturated or unsaturated, substituted or unsubstituted, branched or straight chain hydrocarbyl moiety that may or may not be substituted with hetero atoms; R², R³, and R⁴ are independently selected from the group consisting of H, OH, COOH, wherein upon hydrolysis the precursor compound is capable of producing an active species effective in inhibiting the production of exoprotein from Gram positive bacteria.

The present invention is further directed to an absorbent article for inhibiting the production of exoproteins from Gram positive bacteria. The absorbent article is suitable for insertion into the vagina and comprises an absorbent structure and an effective amount of a precursor compound having the general formula: 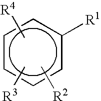
wherein R¹ is 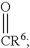
R⁶ is selected from the group consisting of an amino acid, a methyl ester of an amino acid, and an ethyl ester of an amino acid; R², R³, and R⁴ are independently selected from the group consisting of H, OH, COOH, wherein upon hydrolysis the precursor compound is capable of producing an active species effective in inhibiting the production of exoprotein from Gram positive bacteria.

The present invention is further directed to a vaginal tampon for inhibiting the production of exoprotein from Gram positive bacteria. The vaginal tampon comprises an absorbent tampon material and an effective amount of a precursor compound having the general formula: 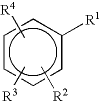
wherein R¹ is 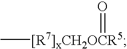
R⁷ is —OCH₂—; X is 0 or 1; R⁵ is a substituted or unsubstituted aromatic ring or a monovalent saturated or unsaturated, substituted or unsubstituted, branched or straight chain hydrocarbyl moiety that may or may not be substituted with hetero atoms; R², R³, and R⁴ are independently selected from the group consisting of H, OH, COOH, wherein upon hydrolysis the precursor compound is capable of producing an active species effective in inhibiting the production of exoprotein from Gram positive bacteria.

The present invention is further directed to a vaginal tampon for inhibiting the production of exoprotein from Gram positive bacteria. The vaginal tampon comprises an absorbent tampon material and an effective amount of a precursor compound having the general formula: 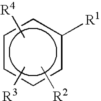
wherein R¹ is 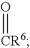
R⁶ is selected from the group consisting of an amino acid, a methyl ester of an amino acid, and an ethyl ester of an amino acid; R², R³, and R⁴ are independently selected from the group consisting of H, OH, COOH, wherein upon hydrolysis the precursor compound is capable of producing an active species effective in inhibiting the production of exoprotein from Gram positive bacteria.

Other features and advantages of this invention will be in part apparent and in part pointed out hereinafter.

### DETAILED DESCRIPTION OF THE PREFERRED EMBODIMENTS

The present invention is generally directed to absorbent articles comprising a precursor compound that upon hydrolysis produces an active species capable of inhibiting the production of exproteins from Gram positive bacteria. Specifically, the present invention relates to absorbent articles comprising a precursor compound formed by linking an aromatic compound to a second compound by an ester or amide bond that can be readily hydrolyzed by enzymatic action once inside of the vagina to produce an active species and a second compound. The active species have been found to substantially inhibit the production of exoproteins, such as TSST-1, from Gram positive bacteria. Typically, the precursor compound itself may inhibit the production of exoproteins from Gram positive bacteria. Additionally, the precursor compounds can be used in combination with surface-active agents such as, for example, myreth-3 myristate, glycerol monolaurate, and laureth-4, to substantially inhibit the production of exoproteins from Gram positive bacteria.

This invention will be described herein in detail in connection with a vaginal tampon, but will be understood by persons skilled in the art to be applicable to other disposable absorbent articles such as sanitary napkins, panty liners, adult incontinence garments, absorbent contraceptive sponges, diapers, wound dressings, medical bandages and other absorbent tampons such as those intended for medical, dental, surgical, and/or nasal use wherein the inhibition of exoproteins from Gram positive bacteria would be beneficial.

As used herein, the phrase “absorbent tampon” generally refers to vaginal tampons, medical tampons, dental tampons, surgical tampons, and nasal tampons. The phrase “absorbent article” generally refers to devices, which absorb and contain body fluids, and more specifically, refers to devices that are placed against or near the skin, or inside of a body cavity, to absorb and contain the various fluids discharged from the body. The term “disposable” is used herein to describe absorbent articles that are not intended to be laundered or otherwise restored or reused as an absorbent article after a single use. Examples of such disposable absorbent articles include, but are not limited to, health care related products including bandages and tampons such as those intended for medical, dental, surgical and/or nasal use; personal care absorbent products such as feminine hygiene products (e.g., sanitary napkins, panty liners, and vaginal tampons), diapers, training pants, incontinence products and the like, wherein the inhibition of the production of exoproteins from Gram positive bacteria would be beneficial.

Vaginal tampons suitable for use with the present invention are typically made of absorbent materials such as absorbent fibers, including natural and synthetic fibers, compressed into a unitary body of a size that may easily be inserted into the vaginal cavity. Suitable fibers include, for example, cellulosic fibers such as cotton and rayon. Fibers may be 100% cotton, 100% rayon, a blend of cotton and rayon, or other materials known to be suitable for tampon use.

Vaginal tampons are typically made in an elongated cylindrical form in order that they may have a sufficiently large body of material to provide the required absorbing capacity, but may be made in a variety of shapes. The tampon may or may not be compressed, although compressed types are generally preferred. The tampon may be made of various fiber blends including both absorbent and nonabsorbent fibers, which may or may not be enclosed in a cover or wrapper. Typically, the cover or wrapper can be formed from a nonwoven material such as a polyolefin, particularly polypropylene or polyethylene. A suitable material is a spunbond material. The cover or wrapper is beneficial in assuring that the fibers of the tampon do not directly contact the inner walls of a woman's vagina. This assures that no fibers will be left behind in the vagina after the tampon has been removed. The cover can be tucked into distally spaced ends of the tampon so as to completely surround and enclose the fibers. The cover or wrapper can also be constructed from a heat-sealable material to assist in bonding it to the fibers, such as by heat and/or pressure. Suitable methods and materials for the production of tampons are well known to those skilled in the art.

In one embodiment, a suitable tampon for use in the present invention has a cover or wrapper. Typically, the weight of the tampon having a cover or wrapper will depend upon the level of absorbency of the tampon. For example, a more absorbent tampon will be heavier than a less absorbent tampon. Typically, a regular absorbency tampon with a cover or wrapper will weigh from about 1.77 grams to about 2.67 grams, suitably about 2.22 grams; a super absorbency tampon with a cover or wrapper will weigh from about 2.67 grams to about 3.57 grams, suitably about 3.12 grams; a super plus absorbency tampon with a cover or wrapper will weigh from about 3.67 grams to about 4.97 grams, suitably about 4.32 grams.

Tampons come in a variety of sizes. In another embodiment, a tampon for use in the present invention does not have a cover or wrapper. Typically, a regular absorbency tampon without a cover or wrapper suitable for the present invention will weigh from about 1.60 grams to about 2.50 grams, suitably about 2.05 grams; a super absorbency tampon without a cover or wrapper will weigh from about 2.49 grams to about 3.39 grams, suitably about 2.94 grams; a super plus absorbency tampon without a cover or wrapper will weigh from about 3.49 grams to about 4.79 grams, suitably about 4.14 grams.

As stated above, the absorbent articles of the present invention comprise an effective amount of a precursor compound that upon hydrolysis, produces an active species that can substantially inhibit the production of exoprotein by Gram positive bacterium and, specifically, the production of TSST-1 from S. aureus bacterium. As used herein, the term “precursor compound” means a compound that is introduced into and/or onto an absorbent article that is capable of undergoing hydrolysis inside and/or adjacent to the vagina to produce an active species capable of inhibiting the production of exoproteins from Gram positive bacteria. The precursor compounds are formed by linking an aromatic compound to a second compound with an ester or amide bond. The ester or amide bond contained in the precursor compound is hydrolyzed by enzymes, such as lipase, esterase and/or amidase, which are produced by bacteria found in the natural vaginal flora, resulting in an active species that can inhibit exoprotein production from Gram positive bacteria. Along with the active species produced, the hydrolysis reaction produces a second compound that is not critical to the function of the active species. In some embodiments, the second compound will be identical or similar to compounds naturally occurring in the human body. Although, as noted above, the second compound is not critical, the precursor compound should be designed such that upon hydrolysis the formed second compound is not substantially harmful to the vagina or the bacteria located therein.

During the hydrolysis process, the precursor compound is slowly broken down into the active species and the secondary compound; and as noted above, both the precursor compound and the active species can inhibit the production of exoproteins from Gram positive bacteria. This property is advantageous as it allows for long-lasting continuous inhibition of exoprotein production by Gram positive bacteria. The precursor compounds of the present invention can inhibit the production of exoproteins prior to hydrolysis; and then as the precursor compounds are hydrolyzed, the active species are produced, which can further inhibit exoprotein production.

The precursor compounds described herein and suitable for introduction into and/or onto an absorbent article are substantially stable in and/or on the absorbent article both throughout the manufacturing process and during shelf storage. Stated another way, the precursor compounds are not easily volatized off of or out of the absorbent article during high temperature manufacturing processes or during shipment and storage. This property of the precursor compound is highly desirable as it is important for the precursor compound to remain in or on the absorbent product for ultimate use by the consumer. As noted above, volatility of active compounds from absorbent articles has been problematic in the past and can result in an absorbent article devoid of active compound.

Without being bound to a particular theory, it is believed that the precursor compounds of the present invention may remain in or on the absorbent articles in an increased amount as compared to prior ingredients due to their increased molecular weight. Although the precursor compounds of the present invention have a generally higher molecular weight as compared to some active ingredients utilized in the past, they are suitably hydrolyzed in the body to produce highly desirable active species such as benzyl alcohol and benzoic acid, which are highly effective in inhibiting the production of exoprotein by Gram positive bacteria.

In addition to having a reduced volatility and being capable of remaining in and/or on an absorbent product throughout manufacturing, shipping, and storage, the precursor compounds described herein, along with the hydrolyzed active species and secondary compounds produced in the body, do not kill a substantial amount of naturally occurring bacteria found in the vagina. This property is significant as the complete, or substantially complete, non-specific killing of bacteria located in and around the vagina can be very harmful for the host as natural flora are required to maintain a healthy vagina. In contrast to agents such as antibacterials or antivirals that non-specifically kill all bacteria in the vagina, the precursor compounds and hydrolyzed compounds produced in and around the vaginal cavity do not have a substantial killing effect on bacteria at the concentration incorporated into the product; but the active species produced by hydrolysis can substantially inhibit the production of exotoxins from Gram positive bacteria.

In one embodiment of the present invention, the precursor compounds have the general chemical structure: 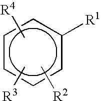
wherein R¹ is 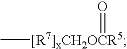
R⁷ is —OCH₂—; X is 0 or 1; R⁵ is a substituted or unsubstituted aromatic ring or a monovalent saturated or unsaturated, substituted or unsubstituted, branched or straight chain hydrocarbyl moiety that may or may not be substituted with hetero atoms; and R², R³, and R⁴ are independently selected from the group consisting of H, OH, COOH.

As noted above, the hydrocarbyl moieties described herein include both straight chain and branched chain hydrocarbyl moieties that may or may not be substituted with various substituents such as, for example, hydroxyl groups. Additionally, the hydrocarbyl moiety may or may not be interrupted with hetero atoms. Hetero atoms that can interrupt the hydrocarbyl moiety include, for example, oxygen, nitrogen, and sulfur. In one embodiment, R⁵ is a monovalent saturated, substituted or unsubstituted, branched or straight chain hydrocarbyl moiety that may or may not be substituted with hetero atoms having from 1 to 15 carbon atoms, more suitably from 1 to 12 carbon atoms.

Some specific precursor compounds having an ester linkage and suitable for use in the above described embodiment of the present invention can be found in Table 1:

TABLE 1

| Compound | Formula |
|---|---|
| Benzyl (s)-(−)- lactate | 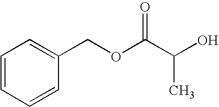 |
| Benzyl ethyl malonate | 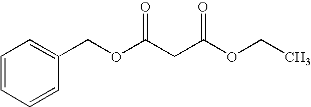 |
| Benzyl- laurate | 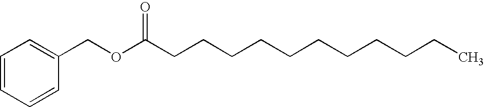 |
| Benzyl benzoate | 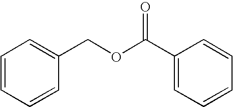 |
| Benzyl paraben | 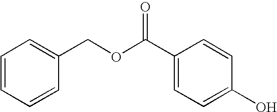 |
| Benzyl salicylate | 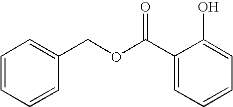 |
| Phenoxy- ethyl paraben | 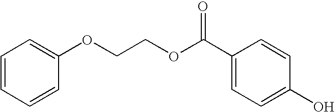 |

The above compounds can be used alone or in combinations thereof.

As noted herein, enzymes produced by bacteria such as S. aureus found in the vaginal flora along with enzymes naturally occurring in the menstrual fluid can hydrolyze the precursor compounds described herein to produce an active species and a second compound. For example, the enzyme esterase can react with the ester linkage of the precursor compounds described above to form the active species benzyl alcohol and a hydrocarbon. Benzyl alcohol has been found to substantially inhibit exoproteins from Gram positive bacteria without substantially eliminating the bacteria.

In another embodiment, the precursor compounds have the general chemical structure: 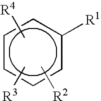
wherein R¹ is 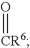
R⁶ is selected from the group consisting of an amino acid, a methyl ester of an amino acid, and an ethyl ester of an amino acid; R², R³, and R⁴ are independently selected from the group consisting of H, OH, COOH.

Amino acids are organic compounds containing an amino group and a carboxylic acid group. Suitable amino acids that can be used for R⁶ are any of the twenty amino acids found naturally in the human body. More particularly, amino acids for use in the present invention suitably include, for example, valine, leucine, cysteine, and combinations thereof.

Suitable compounds for use in this embodiment can be found in Table 2 below:

TABLE 2

| Compound | Formula |
|---|---|
| N-Benzoyl-DL-Valine | 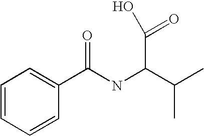 |
| N-Benzoyl-DL-Leucine | 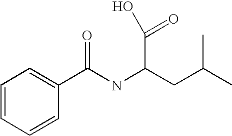 |
| N-Benzoyl-DL-Cysteine | 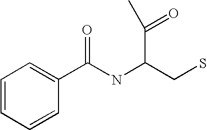 |

The above compounds described herein can be used alone or in combinations thereof.

As described above, enzymes produced by bacteria such as S. aureus found in the vaginal flora along with enzymes naturally occurring in the menstrual fluid can hydrolyze the precursor compounds containing the amino acid. For example, the enzyme amidase can react with the precursor compound and break the amide bonds of the compounds in this embodiment to release benzoic acid and an amino acid. Like benzyl alcohol discussed above, benzoic acid has been found to substantially inhibit exoproteins from Gram positive bacteria without substantially eliminating the naturally occurring flora.

In accordance with the present invention, the absorbent article contains an effective amount of the precursor compound such that upon hydrolysis of the precursor compound there is a sufficient amount of active agent produced to substantially inhibit the formation of exoproteins, such as TSST-1, when the absorbent article is exposed to S. aureus bacteria. Several methods are known in the art for testing the effectiveness of potential inhibitory agents, such as benzyl alcohol or benzoic acid, on the inhibition of the production of TSST-1 in the presence of S. aureus. One such preferred method is set forth in Example 1. When tested in accordance with the methodology set forth herein, preferably, the active species produced from the hydrolysis of the precursor compound reduces the formation of TSST-1 by S. aureus by at least about 40%, more preferably by at least about 50%, still more preferably by at least about 60%, still more preferably by at least about 70%, still more preferably by at least about 80%, still more preferably by at least about 90%, and still more preferably by at least about 95%. Additionally, as noted above, the precursor compound may also inhibit the production of exoproteins from Gram positive bacteria in some embodiments. The test procedure set forth in Example 1 can also be used to measure the amount of inhibition by the precursor compound. In some embodiments, the precursor compound may reduce the production of TSST-1 by at least about 50%, preferably at least about 80%.

In accordance with the present invention, the absorbent products comprise an effective amount of precursor compound such that, upon use, the precursor compound and/or the active species produced therefrom in and around the vagina as discussed herein can substantially inhibit the production of exoprotein from Gram positive bacteria. Generally, absorbent articles will comprise from about 0.15% (by weight of the absorbent structure) to about 2.0% (by weight of the absorbent structure) precursor compound. These percentages are commonly referred to as “add on weight percentages.” In a desirable embodiment, the absorbent products will comprise from about 0.17% (by weight of the absorbent structure) to about 1.7% (by weight of the absorbent structure) precursor compound.

In one specific embodiment, a vaginal tampon as described herein without a cover or wrapper material comprises an effective amount of precursor compound such that, upon use, the precursor compound and/or the active species produced therefrom in or around the vagina as discussed herein can substantially inhibit the production of exoprotein from Gram positive bacteria. Generally, the vaginal tampon without a cover or wrapper will comprise from about 0.15% (by weight of the absorbent tampon material) to about 2.0% (by weight of the absorbent tampon material) precursor compound. In a desirable embodiment, the vaginal tampon will comprise from about 0.17% (by weight of the absorbent tampon material) to about 1.7% (by weight of the absorbent tampon material) precursor compound.

In another specific embodiment, a vaginal tampon as described herein is provided having a cover or wrapper material that comprises a suitable amount of precursor compound such that, upon use, the precursor compound and/or the active species produced therefrom in or around the vagina as discussed herein can substantially inhibit the production of exoprotein from Gram positive bacteria. The precursor compound is introduced directly into or onto the cover or wrapper material as opposed to being introduced into or onto the absorbent substrate of the vaginal tampon. In a desirable embodiment, the cover or wrapper material of the vaginal tampon will comprise from about 2.6% (by weight of the cover or wrapper material) to about 35.0% (by weight of the cover or wrapper material) precursor compound. More desirably, the cover or wrapper material of the vaginal tampon will comprise from about 2.95% (by weight of the cover or wrapper material) to about 29.5% (by weight of the cover or wrapper material) precursor compound.

In a preferred embodiment of the present invention, the precursor compounds described herein can be introduced into and/or onto the absorbent product in combination with one or more surface-active agents to further reduce the production of exoproteins such as TSST-1 without significantly eliminating the beneficial bacterial flora. The surface-active agents used in combination with the precursor compounds may also act as lubricants and/or emollients to further improve product performance. When used in combination with a vaginal tampon, the surface-active agents can further aid in the removal of a “dry tampon”. In one embodiment including a vaginal tampon, a suitable surface-active agent is myreth-3-myristate, which is commercially sold as CETIOL 1414 by Kraft Chemical Corp. (Melrose Park, Ill.). Other suitable surface-active agents for the present invention include, for example, glycerol monolaurate and laureth-4.

In accordance with the present invention, the absorbent products can comprise a suitable amount of surface-active agents such that, upon use, the surface-active agents can further inhibit the production of exoprotein from Gram positive bacteria. Generally, the absorbent articles will comprise from about 0.4% (by weight of the absorbent structure) to about 1.1% (by weight of the absorbent structure) surface-active agent. In one specific embodiment, the absorbent product is a vaginal tampon having a cover and will comprise about 0.75% (by weight of the total tampon including a cover) surface-active agent.

In another embodiment, the surface-active ingredient can be introduced directly into or onto the cover or wrapper material as opposed to being introduced into or onto the absorbent substrate of the vaginal tampon. Typically, the cover or wrapper material of the vaginal tampon will comprise about 13% (by weight of the cover material) surface-active agent.

Additionally, the precursor compounds described herein can be introduced into and/or onto the absorbent product in combination with one or more secondary agents to further reduce the production of exoproteins such as TSST-1 without significantly eliminating the beneficial bacterial flora. Suitable examples of secondary agents useful in the present invention include agents selected from the group consisting of: compounds with an ether, ester, amide, glycosidic, or amine bond linking a C₈-C₁₈ fatty acid to an aliphatic alcohol.

In one embodiment, the precursor compound described herein can be used in combination with ester comopunds having the general formula: 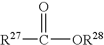
wherein R²⁷ is a straight or branched alkyl or straight or branched alkenyl having from 8 to about 18 carbon atoms and R²⁸ is selected from the group consisting of an alcohol, a polyhydric alcohol, and an ethoxylated alcohol. As used herein, the term “polyhydric” refers to the presence in a chemical compound of at least two hydroxyl (OH) groups. Suitable compounds include glyceryl monolaurate, glyceryl dilaurate, myreth-3-myristate, and mixtures thereof.

In another embodiment, the precursor compound described herein can be used in combination with ether compounds having the general formula:
R¹⁰—O—R¹¹
wherein R¹⁰ is a straight or branched alkyl or straight or branched alkenyl having from 8 to about 18 carbon atoms and R¹¹ is selected from the group consisting of an alcohol, an ethoxylated alcohol, a polyalkoxylated sulfate salt and a polyalkoxylated sulfosuccinate salt. Suitable compounds include laureth-3, laureth-4, laureth-5, PPG-5 lauryl ether, 1-0-dodecyl-rac-glycerol, sodium laureth sulfate, potassium laureth sulfate, disodium laureth (3) sulfosuccinate, dipotassium laureth (3) sulfosuccinate, and polyethylene oxide (2) sorbital ether.

In another embodiment, the precursor compounds described herein can be used in combination with an alkyl polyglycoside compound. Suitable alkyl polyglycosides for use in combination with the precursor compounds include alkyl polyglycosides having the general formula:
H—(Zn)—O—R¹⁴
wherein Z is a saccharide having 5 or 6 carbon atoms, n is a whole number from 1 to 6, and R¹⁴ is a linear or branched alkyl group having from about 8 to about 18 carbon atoms. Glucopon 220, 225, 425, 600, and 625 (all commercially available from Henkel Corporation, Ambler, Pa.) and TL 2141 (commercially available from ICI Surfactants, Wilmington, Del.) are suitable alkyl polyglycosides for use in combination with the precursor compounds of the present invention.

In another embodiment, the precursor compounds described herein can be used in combination with an amide containing compound having the general formula: 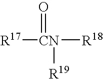
wherein R¹⁷, inclusive of the carbonyl carbon, is an alkyl group having 8 to 18 carbon atoms, and R¹⁸ and R¹⁹ are independently selected from hydrogen or an alkyl group having from 1 to about 12 carbon atoms which may or may not be substituted with groups selected from ester groups, ether groups, amine groups, hydroxyl groups, carboxyl groups, carboxyl salts, sulfonate groups, sulfonate salts, and mixtures thereof. Preferred amide compounds for use in combination with the precursor compounds described herein include sodium lauryl sarcosinate, lauramide monoethanolamide, lauramide diethanolamide, lauramidopropyl dimethylamine, disodium lauramido monoethanolamide sulfosuccinate, and disodium lauroamphodiacetate.

In another embodiment, the precursor compounds described herein can be used in combination with amine compounds having the general formula: 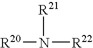
wherein R²⁰ is an alkyl group having from about 8 to about 18 carbon atoms and R²¹ and R²² are independently selected from the group consisting of hydrogen and alkyl groups having from 1 to about 18 carbon atoms and which can have one or more substitutional moieties selected from the group consisting of hydroxyl, carboxyl, carboxyl salts, and imidazoline. Preferred amine compounds for use with the precursor compounds described herein include triethanolamide laureth sulfate, lauramine, lauramino propionic acid, sodium lauriminodipropionic acid, lauryl hydroxyethyl imidazoline, and mixtures thereof.

In another embodiment, the amine compound can be an amine salt having the general formula: 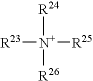
wherein R²³ is an anionic moiety associated with the amine and is derived from an alkyl group having from 8 to about 18 carbon atoms and R²⁴, R², and R²⁶ are independently selected from the group consisting of hydrogen and alkyl group having from 1 to about 18 carbon atoms and which can have one or more substitutional moieties selected from the group consisting of hydroxyl, carboxyl, carboxyl salts, and imidazoline. R²⁴, R²⁵, and R²⁶ can be saturated or unsaturated. A preferred compound illustrative of an amine salt is TEA laureth sulfate.

Amounts of secondary compounds described herein to be added to the absorbent articles to further reduce the production of TSST-1 have been found to be from about 0.15% (by weight of the absorbent structure) to about 2.0% (by weight of the absorbent structure) secondary compound. More suitably, the absorbent products comprise from about 0.17% (by weight of the absorbent structure) to about 1.7% (by weight of the absorbent structure) secondary compound.

The precursor compounds of the present invention can be prepared and applied to the absorbent article in any suitable form, but are preferably prepared in forms including, without limitation, aqueous solutions, lotions, balms, gels, salves, ointments, boluses, suppositories, and the like.

The precursor compounds may be applied to the absorbent article using conventional methods for applying a chemical agent to the desired absorbent article. For example, unitary tampons without separate wrappers may be sprayed with a solution containing the desired concentration of the precursor compound and then can be air dried, if necessary, to remove any volatile solvents. For compressed tampons, it is generally preferable to impregnate the tampon with any chemical compounds before compressing. The precursor compounds when incorporated on and/or into the tampon materials may be fugitive, loosely adhered, bound, or any combination thereof. As used herein, the term “fugitive” means that the composition is capable of migrating through the tampon materials.

Generally, it is not necessary to impregnate the entire absorbent body of the tampon with the precursor compound. Optimum results both economically and functionally can be obtained by concentrating the precursor compound on or near the outer surface of the tampon or other absorbent product where it may be most effective during use.

The precursor compounds may additionally employ one or more conventional pharmaceutically-acceptable and compatible carrier materials useful for the desired application to facilitate absorption into the absorbent product. The carrier can be capable of co-dissolving or suspending the precursor compound used in the absorbent article. Carrier materials suitable for use in the instant invention include those well-known for use in the cosmetic and medical arts as a basis for ointments, lotions, creams, salves, aerosols, suppositories, gels, and the like. For example, one suitable carrier is Cetiol. As discussed above, Cetiol can also act as both a lubricant and an emollient. Other suitable carriers include water, various alcohols, and other organic solvents.

The precursor compounds of the present invention may, in some embodiments, be used in combination with adjunct components conventionally found in pharmaceutical compositions in their art-established fashion and at concentrations that would not alter normal vaginal flora. For example, the compositions may contain additional compatible pharmaceutically active materials for combination therapy, such as supplementary selective antibacterials, antioxidants, anti-parasitic agents, antipruritics, astringents, local anaesthetics, or anti-inflammatory agents.

In one embodiment, the precursor compounds can be microencapsulated in a shell-type material that will dissolve, disintegrate, rupture, or otherwise breakdown upon contact with menses or other vaginal secretions to release the component. With this embodiment, the encapsulation material retards volatilization of the precursor compound until wetted with a bodily secretion, which results in a release of the precursor compound. Such encapsulation can significantly increase the amount of precursor compound present in the product after manufacturing and storage. Suitable microencapsulated shell materials are known in the art and include cellulose-based polymeric materials (e.g., ethyl cellulose), carbohydrate-based materials (e.g., cationic starches and sugars) and materials derived therefrom (e.g., dextrins and cyclodextrins) as well as other materials compatible with human tissues.

The microencapsulation shell thickness may vary and is generally manufactured to allow the encapsulated precursor compound to be covered by a thin layer of encapsulation material, which may be a monolayer or thicker laminate layer, or may be a composite layer. The microencapsulation layer should be thick enough to resist cracking or breaking of the shell during handling or shipping of the product. The microencapsulation layer should be constructed such that humidity from atmospheric conditions during storage, shipment, or wear will not cause a breakdown of the microencapsulation layer and result in a release of the precursor compound.

Microencapsulated compounds applied directly to the absorbent articles should be of a size such that the user cannot feel the encapsulated shell on the skin or mucosa during use. Typically, the capsules have a diameter of no more than about 25 micrometers, and desirably no more than about 10 micrometers. At these sizes, there is no “gritty” or “scratchy” feeling when the compound contacts the skin.

The present invention is illustrated by the following examples which are merely for the purpose of illustration and are not to be regarded as limiting the scope of the invention or manner in which it may be practiced.

### EXAMPLE 1

In this Example, the effect of various test compounds on the growth of S. aureus and production of TSST-1 was determined. The test compound, in the desired concentration (expressed in wt % (w/v)) was placed in 10 mL of a growth medium in a sterile, 50 mL conical polypropylene tube (Sarstedt, Inc., Newton, N.C.).

The growth medium was prepared by dissolving 37 grams of brain heart infusion broth (BHI) (Difco Laboratories, Cockeysville, Md.) in 880 mL distilled water and sterilizing the broth according to the manufacturer's instructions. The BHI was supplemented with 100 mL fetal bovine serum (FBS) (Sigma Chemical Company, St. Louis, Mo.). Ten mL of a 0.021 M sterile solution of hexahydrate of magnesium chloride (Sigma Chemical Company, St. Louis, Mo.) was added to the BHI-FBS mixture. Ten mL of a 0.027 M sterile solution of L-glutamine (Sigma Chemical Company, St. Louis, Mo.) was also added to the BHI-FBS mixture.

Compounds to be tested included N-benzoyl-DL-leucine (Sigma B-1504) and N-benzoyl-DL-valine (Sigma B-6500). Test compounds were received as solids. The solids were dissolved in BHI prepared as described above. The test compounds were added to the growth medium in the amount necessary to obtain the desired final concentration. Cetiol 1414E (Kraft Chemical Corp., Melrose Park, Ill.) was included in the growth medium in some assays at a 10 mM concentration.

In preparation for inoculation of the tubes of growth medium containing the test compounds, an inoculating broth was prepared as follows: S. aureus MN8 was streaked onto a tryptic soy agar plate (TSA; Difco Laboratories, Cockeysville, Md.) and incubated at 35° C. The test organism in this example was obtained from Dr. Pat Schlievert, Department of Microbiology, University of Minnesota Medical School, Minneapolis, Minn. After 24 hours of incubation three to five individual colonies were picked with a sterile inoculating loop and used to inoculate 10 mL of growth medium. The tube of inoculated growth medium was incubated at 35° C. in atmospheric air. After 24 hours of incubation, the culture was removed from the incubator and mixed well on a S/P brand vortex mixer. A second tube containing 10 mL of the growth medium was inoculated with 0.5 mL of the above-described 24 hour old culture and incubated at 35° C. in atmospheric air. After 24 hours of incubation the culture was removed from the incubator and mixed well on a S/P brand vortex mixer. The optical density of the culture fluid was determined in a microplate reader (Bio-Tek Instruments, Model EL309, Winooski, Vt.). The amount of inoculum necessary to give 5×10⁶ CFU/mL in 10 mL of growth medium was determined using a previously prepared standard curve.

This Example included tubes of growth medium with varying concentrations of test compounds, varying concentrations of test compounds and Cetiol 1414E, and tubes of growth medium without test compounds (control). Each tube was inoculated with the amount of inoculum determined as described above. The tubes were capped with foam plugs (IDENTI-PLUG plastic foam plugs, Jaece Industries, purchased from VWR Scientific Products, South Plainfield, N.J.). The tubes were incubated at 35° C. in atmospheric air containing 5% by volume CO₂. After 24 hours of incubation, the tubes were removed from the incubator, the culture fluid was assayed for the number of colony forming units of S. aureus, and the culture fluid was prepared for the analysis of TSST-1 as described below.

The number of colony forming units per mL after incubation was determined using standard plate count procedures. In preparation for analysis of TSST-1, the culture fluid broth was centrifuged at 2500 rpm at 2-10° C. for 15 minutes and the supernatant subsequently filter sterilized through a FISHERBRAND 0.45 μm MCE filter, 0.2 μM pore size. The resulting fluid was frozen at −70° C. in a FISHERBRAND 12×75 mm polystyrene culture tube (Fisher Scientific, Pittsburgh, Pa.).

The amount of TSST-1 per mL was determined by a non-competitive, sandwich enzyme-linked immunoabsorbent assay (ELISA). The method employed was as follows: four reagents, TSST-1 (#TT-606), rabbit polyclonal anti-TSST-1 IgG (LTI-101), rabbit polyclonal anti-TSST-1 IgG conjugated to horseradish peroxidase (#LTC-101), and normal rabbit serum (NRS) certified anti-TSST-1 free (#NRS-10) were purchased from Toxin Technology, Inc. (Sarasota, Fla.). A 10 μg/mL solution of the polyclonal rabbit anti-TSST-1 IgG was prepared in phosphate buffered saline (PBS) (pH 7.4). The PBS was prepared from 0.016 M NaH₂PO₄, 0.004 M NaH₂PO₄—H₂O, 0.003 M KCl and 0.137 M NaCl, all available from Sigma Chemical Company (St. Louis, Mo.). One hundred microliters of the polyclonal rabbit anti-TSST-1 IgG solution was pipetted into the inner wells of polystyrene microplates, (Nunc-Denmark, Catalogue Number #439454). The plates were covered and incubated at room temperature overnight. Unbound anti-toxin was removed by draining until dry.

TSST-1 was diluted to 10 ng/mL with phosphate buffered saline (PBS) (pH 7.4) containing 0.05% (vol/vol) Tween-20 (PBS-Tween) (Sigma Chemical Company, St. Louis, Mo.) and 1% (vol/vol) NRS and incubated at 4° C. overnight. Samples of the culture fluid and the TSST-1 reference standard were assayed in triplicate.

One hundred microliters of a 1% (wt/vol) solution of the sodium salt of casein (Sigma Chemical Company, St. Louis, Mo.) in PBS were pipetted into the inner wells of polystyrene microplates. The plates were covered and incubated at 35° C. for one hour. Unbound BSA was removed by 3 washes with PBS-Tween. TSST-1 reference standard (10 ng/mL) treated with NRS, test samples treated with NRS, and reagent controls were pipetted in 200 μL volumes to their respective wells on the first and seventh columns of the plate. One hundred microliters of PBS-Tween were added to the remaining wells. The TSST-1 reference standard and test samples were then serially diluted 5 times in the PBS-Tween by transferring 100 microliters from well-to-well. The samples were mixed prior to transfer by repeated aspiration and expression. Samples of the test samples and the TSST-1 reference standard were assayed in triplicate. This was followed by incubation for 1.5 hours at 35° C. and five washes with PBS-T and three washes with distilled water to remove unbound toxin. The rabbit polyclonal anti-TSST-1 IgG conjugated to horseradish peroxidase was diluted according to manufacturer's instructions and 50 microliters was added to each microtiter well, except well A-1, the conjugate control well. The plates were covered and incubated at 35° C. for one hour.

Following incubation the plates were washed five times in PBS-Tween and three times with distilled water. Following the washes, the wells were treated with 100 microliters of a horseradish peroxidase substrate buffer consisting of 5 mg of o-phenylenediamine and 5 μL of 30% hydrogen peroxide (both available from Sigma Chemical Company, St. Louis, Mo.) in 11 mL of citrate buffer (pH 5.5). The citrate buffer was prepared from 0.012 M anhydrous citric acid and 0.026 M dibasic sodium phosphate both available from Sigma Chemical Company (St. Louis, Mo.). The plates were incubated for 15 minutes at 35° C. The reaction was stopped by the addition of 50 microliters of a 5% sulfuric acid solution. The intensity of the color reaction in each well was evaluated using the Bio-Tek Model EL309 microplate reader (OD 490 nm). TSST-1 concentrations in test samples were determined from the reference toxin regression equation derived during each assay procedure. The efficacy of the compound in inhibiting the production of TSST-1 is shown in Table 3 below.

TABLE 3

| | Amount | | | | |
|---|---|---|---|---|---|
| Amino acid | test | | | ELISA: TSST-1 | |
| containing | compound | Cetiol | | ng per mL | |
| compound | (wt %) | 1414E | CFU/mL | (% inhibition) | |
| None | | None | 2.63E+08 | 178.4 | (N/A) |
| N-benzoyl-DL- | 0.25% | None | 2.69E+08 | 14.6 | (92.8%) |
| leucine | (w/v) | | | | |
| N-benzoyl-DL- | 0.20% | None | 1.61E+08 | 25.1 | (79%) |
| valine | (w/v) | | | | |
| None | | 10 mM | 1.75E+08 | 53.1 | (60%) |
| N-benzoyl-DL- | 0.25% | 10 mM | 2.23E+08 | 7.9 | (95%) |
| leucine | (w/v) | | | | |
| N-benzoyl-DL- | 0.20% | 10 mM | 2.07E+08 | 10.6 | (93.2%) |
| valine | (w/v) | | | | |

N/A = Not Applicable

In accordance with the present invention, the data in Table 3 shows that S. aureus MN8, when compared to the control, produced less TSST-1 in the presence of the amino acid containing test compounds. At the concentrations tested, these compounds reduced the amount of toxin produced by 79% to 93%. Also, the data shows that S. aureus MN8, when compared to the control, produced less TSST-1 in the presence of the amino acid containing test compounds when combined with Cetiol 1414E. At the concentrations tested, these compounds, when combined with Cetiol 1414E, reduced the amount of toxin produced by 93% to 95%. However, although the amount of toxin produced was significantly reduced under these conditions, there was minimal, if any, effect on the growth of S. aureus.

### EXAMPLE 2

In this Example, the effect of benzyl alcohol (Aldrich 40,283-4) and benzyl ethyl malonate (Aldrich 30,069-1) (Sigma Chemical Corporation, St. Louis, Mo.) on the growth of S. aureus and the production of TSST-1 was determined. The effect of the test compounds tested in Example 2 was determined by placing the desired concentration, expressed in % (v/v), in 10 mL of a growth medium as described in Example 1. The test compounds were then tested and evaluated as in Example 1, except that each test was carried out in quadruplicate. The results shown represent an average of the four values. The effect of the test compounds on growth of S. aureus MN8 and on the production of TSST-1 is shown in Table 4 below.

TABLE 4

| | Amount | | | | |
|---|---|---|---|---|---|
| | Test | | | ELISA: TSST-1 | |
| | Compound | Cetiol | | ng per mL | |
| Compound | (%(v/v)) | 1414E | CFU/mL | (% inhibition) | |
| None | | None | 7.36E+08 | 338 | (N/A) |
| Benzyl alcohol | 0.3% | None | 7.52E+08 | 50 | (85%) |
| Benzyl ethyl | 0.8% | None | 3.78E+08 | 19 | (94%) |
| malonate | | | | | |
| None | | 10 mM | 2.61E+08 | 70 | (79%) |
| Benzyl alcohol | 0.3% | 10 mM | 5.02E+08 | 4 | (99%) |
| Benzyl ethyl | 0.8% | 10 mM | 1.51E+08 | 6 | (98%) |
| malonate | | | | | |

N/A = Not Applicable

At the concentrations tested, both the precursor compound, benzyl ethyl malonate, and the active species, benzyl alcohol, reduced the amount of toxin produced by 85% to 94%. Also, the data shows that S. aureus MN8, when compared to the control, produced less TSST-1 in the presence of benzyl alcohol or benzyl ethyl malonate when combined with Cetiol 1414E (myreth-3-myristate). At the concentrations tested, these compounds, when combined with Cetiol 1414E, reduced the amount of toxin produced by 98% to 99%.

Statistical analysis of the treatments was performed using pairwise comparison on Least Squares Means in an Analysis of Variance context. The pairwise comparisons were the equivalents of standard T Tests. The results of the comparisons showed that growth in the presence of Cetiol alone resulted in significantly greater inhibition of the growth of S. aureus than growth in its absence or growth in the presence of benzyl alcohol alone or benzyl ethyl malonate with or without Cetiol. Growth in the presence of benzyl ethyl malonate and the combinations of Cetiol with benzyl alcohol or benzyl ethyl malonate resulted in significantly decreased toxin production when compared to growth in the presence of Cetiol alone or no additives. However, although the amount of toxin produced was significantly reduced under these conditions, there was minimal, if any, effect on the growth of S. aureus.

### EXAMPLE 3

In this Example, the effect of benzyl (s)-(−)-lactate (Aldrich 42,484-6) (Sigma Chemical Corporation, St. Louis, Mo.) on the growth of S. aureus and the production of TSST-1 was determined. The effect of the test compounds was determined by placing the desired concentration, expressed in % (v/v), in 10 mL of a growth medium as in Example 1. The compounds were then tested and evaluated as in Example 1, except that each test was carried out in quadruplicate. The results shown represent an average of the four values. The effect of the test compounds on the growth of S. aureus MN8 and on the production of TSST-1 is shown in Table 5 below.

TABLE 5

| | Amount | | | | |
|---|---|---|---|---|---|
| | Test | | | ELISA: TSST-1 | |
| | Compound | Cetiol | | Nanograms/mL | |
| Compound | (%(v/v)) | 1414E | CFU/mL | (% inhibition) | |
| None | | None | 4.60E+08 | 604 | (N/A) |
| Benzyl (s)- | 0.3% | None | 4.86E+08 | 18 | (96.9%) |
| (−)-lactate | | | | | |
| None | | 10 mM | 2.48E+08 | 61 | (89.9%) |
| Benzyl (s)- | 0.3% | 10 mM | 1.48E+08 | 5 | (99.2%) |
| (−)-lactate | | | | | |

N/A = Not Applicable

At the concentration tested, benzyl (s)-(−)-lactate reduced the amount of toxin produced by 97%. Also, the data shows that S. aureus MN8, when compared to the control, produced less TSST-1 in the presence of benzyl (s)-(−)-lactate when combined with Cetiol 1414E (myreth-3-myristate). At the concentration tested, benzyl lactate, when combined with Cetiol 1414E, reduced the amount of toxin produced by 99%.

Statistical comparisons of the treatments were performed by the method discussed in Example 2. The results of the comparisons showed that growth in the presence of Cetiol alone or Cetiol and benzyl (s)-(−)-lactate resulted in significantly greater inhibition of the growth of S. aureus than growth in the absence of Cetiol or growth in the presence of benzyl (s)-(−)-lactate alone. Growth in the presence of benzyl (s)-(−)-lactate and the combinations of Cetiol with benzyl (s)-(−)-lactate resulted in significantly decreased toxin production when compared to growth in the presence of Cetiol alone or no additives.

### EXAMPLE 4

In this Example, the effect of benzyl (s)-(−)-lactate in combination with the surface-active agent Cetiol (myreth-3-myristate) was tested using a 5×4 rboard experimental design. This allowed the ion of the interaction of the two test compounds on owth of S. aureus and the production of TSST-1.

Five concentrations of benzyl (s)-(−)-lactate (0.00, 0.06%, 0.13%, 0.25%, and 0.50%) were combined with four concentrations of Cetiol 1414E (10 mM, 5 mM, 2.5 mM, and 0.0 mM) in a twenty tube array. For example, tube #1 contained 0.0 mM Cetiol 1414E and 0.0% benzyl (s)-(−)-lactate (w/v) in 10 mL of growth medium (as prepared in Example 1). Each of the tubes #1-#20 contained a unique combination of benzyl (s)-(−)-lactate and Cetiol. The solutions were tested and evaluated as in Example 1. The effect of the test compounds on the growth of S. aureus MN8 and on the production of TSST-1 is shown in Table 6 below.

TABLE 6

| | Benzyl | | | | |
|---|---|---|---|---|---|
| | (s)-(-)- | | | ng TSST-1 | |
| Cetiol | Lactate | CFU/mL | ng TSST-1 | per 10⁸ | |
| mM | (wt %) | ×10⁷ | per mL | CFU | % Reduction |
| 0 | 0 | 15.3 | 390 | 255 | N/A |
| 0 | 0.5 | 20.0 | 49 | 24 | 90% |
| 0 | 0.25 | 24.6 | 24 | 10 | 96% |
| 0 | 0.13 | 18.0 | 66 | 37 | 86% |
| 0 | 0.06 | 18.3 | 124 | 68 | 73% |
| 2.5 | 0 | 22.5 | 148 | 66 | 74% |
| 2.5 | 0.5 | 1.5 | 3 | 19 | 93% |
| 2.5 | 0.25 | 15.4 | 8 | 5 | 98% |
| 2.5 | 0.13 | 26.2 | 47 | 18 | 93% |
| 2.5 | 0.06 | 20.4 | 51 | 25 | 90% |
| 5.0 | 0 | 25.6 | 203 | 79 | 69% |
| 5.0 | 0.5 | 2.30 | 2 | 7 | 97% |
| 5.0 | 0.25 | 12.0 | 7 | 6 | 98% |
| 5.0 | 0.13 | 18.3 | 19 | 11 | 96% |
| 5.0 | 0.06 | 26.2 | 28 | 11 | 96% |
| 10.0 | 0 | 17.4 | 57 | 33 | 87% |
| 10.0 | 0.5 | 6.1 | 2 | 4 | 99% |
| 10.0 | 0.25 | 14.7 | 8 | 6 | 98% |
| 10.0 | 0.13 | 20.0 | 16 | 8 | 97% |
| 10.0 | 0.06 | 22.6 | 41 | 18 | 93% |

N/A = Not Applicable

At every concentration of Cetiol 1414E, benzyl (s)-(−)-lactate increases the inhibition of TSST-1 production. The effect appears to be additive.

### EXAMPLE 5

In this Example, the effect of benzyl laurate (Penta Manufacturing, Fairfield, N.J.) on the growth of S. aureus MN8 and the production of TSST-1 was determined. The effect of the test compound was determined by placing the desired concentration, expressed in % (v/v), in 10 mL of a growth medium as in Example 1. The compounds were then tested and evaluated as in Example 1. The effect of benzyl laurate on growth of S. aureus MN8 and on the production of TSST-1 is shown in Table 7 below.

TABLE 7

| | Amount | | | | |
|---|---|---|---|---|---|
| | Test | | | ELISA: TSST-1 | |
| | Compound | Cetiol | | ng per mL | |
| Compound | (%(v/v)) | 1414E | CFU/mL | (% inhibition) | |
| None | | None | 4.10E+08 | 450 | (N/A) |
| Benzyl laurate | 0.4% | None | 4.05E+08 | 225 | (62%) |
| Benzyl laurate | 0.6% | None | 3.80E+08 | 55 | (91%) |
| None | | 10 mM | 2.05E+08 | 153 | (79%) |
| Benzyl laurate | 0.4% | 10 mM | 2.05E+08 | 60 | (90%) |
| Benzyl laurate | 0.6% | 10 mM | 3.05E+08 | 83 | (86%) |

N/A = Not Applicable

At the concentrations tested, benzyl laurate reduced the amount of toxin produced by 62% to 91%. At the concentrations tested, benzyl laurate, when combined with Cetiol 1414E (myreth-3-myristate), reduced the amount of toxin produced by 86% to 90%.

### EXAMPLE 6

In this Example, commercial tampons (KOTEX super absorbency, Kimberly-Clark Worldwide, Inc., Neenah, Wis.) were treated with benzyl alcohol to determine the effect of the treated tampon on the growth of S. aureus MN8 and the production of TSST-1. As stated above, the commercial tampon will contain about 0.75% (by weight of the total tampon having a cover) Cetiol (myreth-3-myristate). In preparation for the experiment, the string of the tampon was cut off and the pledget was placed into a sterile, capped 50 mL polystyrene test tube with the string end down.

The pledgets were inoculated with 5 mL of five concentrations (0.0, 0.3%, 0.15%, 0.075%, and 0.03%) of benzyl alcohol dissolved in Brain Heart Infusion Broth (BHI). The tampon pledgets were left to sit at room temperature for one hour.

Each pledget was then inoculated with 5.5 mL of an inoculating broth containing 5×10^{6±1×10}⁶ CFU/mL of S. aureus MN8 to achieve a final volume of 10.5 mL. The tubes were capped with foam plugs (IDENTI-PLUG plastic foam plugs, Jaece Industries, purchased from VWR Scientific Products, South Plainfield, N.J.) and incubated at 37° C. for 24 hours. The pledgets were removed from the incubator and individually placed into sterile STOMACHER bags (Seward Ltd., Norfolk, United Kingdom), which contained 50 mL sterile BHI. The pledgets and fluid were then stomached or blended in the bags. Aliquots of fluid were removed from the STOMACHER bags and placed into sterile tubes for testing.

Plate count samples were prepared by vortexing the sample, withdrawing 5 mL of the sample and placing the 5 mL in a new sterile 50 mL centrifuge tube. The sample was then sonicated using a Virsonic 600 Ultrasonic Cell Disruptor (Virtis Company, Gardiner, N.Y.) for 15 seconds at 8% output power. When all the samples had been sonicated, the number of colony forming units (CFU) per mL was determined using standard plate count procedures.

In preparation for analysis of TSST-1, the culture fluid broth was centrifuged at 9000 rpm at 4° C. for 5 minutes and the supernatant was subsequently filter sterilized through 0.45 μm MCE filter, 0.2 μM pore size. The resulting fluid was frozen at −70° C. in two 1 mL aliquots in 1.5 mL polypropylene screw cap freezer vials.

The amount of TSST-1 per mL was determined by a non-competitive, sandwich enzyme-linked immunoabsorbent assay (ELISA). The method employed was as follows: four reagents, TSST-1 (#606), rabbit polyclonal anti-TSST-1 IgG (LTI-101), rabbit polyclonal anti-TSST-1 IgG conjugated to horseradish peroxidase (#LTC-101), and normal rabbit serum (NRS) certified anti-TSST-1 free (#NRS-10) were purchased from Toxin Technology, Inc. (Sarasota, Fla.). Sixty-two microliters of #LTI-101 was diluted so that a 1:100 dilution gave an absorbance of 0.4 at 205 nm and subsequently added to 6.5 mL of Na₂CO₃ buffer, pH 7.2, 0.5 M carbonate buffer, pH 9.6, and 100 μL of the solution was pipetted into each of the inner wells of the polystyrene microplates (available from Nunc-Denmark, Catalogue Number #439454). The plates were covered and incubated at 37° C. overnight.

The unbound anti-toxin was removed by four washes in an automatic plate washer with phosphate buffered saline (0.016 M Na₂HPO₄, available from Sigma Chemical Co., St. Louis, Mo.), pH 7.2, and 0.9% (w/v) NaCl (VWR Scientific Products, South Plainfield, N.J.) containing 0.5% (v/v) Tween 20 (Sigma Chemical Co. St. Louis, Mo.). The plates were treated with 100 μL of a 1% (w/v) solution of bovine serum albumin (BSA) fraction V (Sigma Chemical Co., St. Louis, Mo.), in the Na₂CO₃ plus NaHCO₃ buffer described above. The plates were again covered and incubated at 37° C. for one hour. Unbound BSA was removed by six washes of 250 μL PBS-Tween.

The test samples were then treated with normal rabbit serum (10% (v/v) concentration) for 15 minutes at room temperature. The TSST-1 reference standard (serially diluted from 2-20 ng/mL in PBS-Tween) and the NRS treated test samples (serially diluted in PBS-Tween so that the resultant TSST-1 concentration is between 2-20 ng), were pipetted in 100 μL volumes to their respective wells. The samples were then incubated for two hours at 37° C. and unbound toxins were then removed with four washes of 250 μL PBS-Tween. The rabbit polyclonal anti-TSST-1 IgG conjugated to horseradish peroxidase was diluted according to the manufacturer's instructions. The final use dilution of the conjugate was determined by running standard curves of TSST-1 reference standard with the conjugate at undiluted, 1:2, and 1:4 dilutions. The dilution that gave results most comparable to previous lots of conjugate was selected. One hundred μL volumes of this dilution were added to each microtiter well. The plates were covered and incubated at 37° C. for one hour.

Following incubation, the plates were washed six times in 250 μL PBS-Tween. Following the washes, the wells were treated with 100 μL of a horseradish peroxidase substrate solution consisting of 0.015 M sodium citrate, pH 4.0, 0.6 mM 2,2′-Azino-bis-(3-ethylbenzthiazoline-6-sulfonic acid) diammonium salt and 0.009% (v/v) hydrogen peroxide (all available from Sigma Chemical Co., St. Louis, Mo.). The intensity of the color reaction in each well was evaluated over time using a VersaMax Molecular Devices Microplate reader (OD 405 nm) and SoftMax Pro software (both available from Molecular Devices, Inc.). TSST-1 concentrations in the test samples were derived from the reference toxin regression equations for each assay procedure.

The efficacy of benzyl alcohol in inhibiting the production of TSST-1 by S. aureus is shown in Table 8 below.

TABLE 8

| Benzyl alcohol | | TSST-1 ng/mL | |
|---|---|---|---|
| add-on (wt %) | CFU/mL (×10⁸) | (% inhibition) | |
| 0* | 8.17 | 2.66 | (N/A) |
| 0.03% | 6.13 | 2.46 | (8%) |
| 0.075% | 2.83 | 2.22 | (17%) |
| 0.15% | 0.5 | 1.33 | (50%) |

N/A = Not Applicable
*As stated above, this Example uses commercial tampons. As such, this sample contains about 0.75% (by weight of the total tampon having a cover) Cetiol 1414E (myreth-3-myristate).

In accordance with the present invention, the data shows that S. aureus MN8 produced less TSST-1 in the presence of tampons that contain both benzyl alcohol and Cetiol 1414E (myreth-3-myristate) as compared to the control tampons that contain only Cetiol 1414E. At the concentrations tested, benzyl alcohol reduced the amount of toxin produced by 8% to 50%.

### EXAMPLE 7

In this Example, the effect of the growth of S. aureus MN8 on the integrity of various test compounds was determined by measuring the amount of breakdown of the test compounds caused by the enzymes produced by S. aureus MN8 bacteria. The test compounds, in the desired concentrations, were placed in 100 mL of a growth medium in a sterile, 500 mL Corning Fleaker (Fisher Scientific, Pittsbury, Pa.). The growth medium and inoculum were prepared as in Example 1.

Compounds to be tested included 0.2% (v/v) benzyl alcohol (Aldrich 40,283-4), 0.5% (v/v) sodium benzoate, 0.3% (wt/v) benzyl (s)-(−)-lactate (Aldrich 42,484-6), 0.8% (v/v) benzyl ethyl malonate (Aldrich 30,069-1), and 0.3% (wt/v) N-benzoyl-DL-leucine (Sigma B-1504). The test compounds were added to the growth medium in the amount necessary to obtain the desired final concentration.

Each fleaker was inoculated with the amount of inoculum determined as described above. The fleakers were capped with sterile aluminum foil and incubated at 35° C. in atmospheric air in a Lab-Line orbital water bath (available from VWR Scientific Products, McGaw Park, Ill.) at 180 rpm. Fifteen milliliter samples were removed at 3, 6, 9, and 24 hours. The optical density (595 nm) of the culture fluid was determined and the culture fluid collected at 24 hours was assayed for the number of colony forming units of S. aureus MN8 using standard plate count procedures. The test compounds, at the concentrations tested, did not inhibit the growth of S. aureus.

In preparation for analysis of the integrity of the test compounds, the culture fluid was centrifuged at 3000 rpm at 2-10° C. for 15 minutes. The supernatant was filter sterilized through an AUTOVIAL 5 syringeless filter, 0.45 μM pore size (available from Whatman, Inc., Clifton, N.J.). The resulting fluid was frozen at −70° C. in a FISHERBRAND (12 mm×75 mm) polystyrene culture tube (Fisher Scientific, Pittsburgh, Pa.) until chemical analysis could be performed.

Using an Agilent Technologies 5973N GC/MS, analysis was performed on undiluted solutions at 3, 6, 9, and 24 hours to evaluate the ability of S. aureus MN8 to breakdown the test compounds. Based on the analysis, the control sample, which had no test compounds added, comprised mostly acetic acid at all time intervals. The samples containing benzoic acid and benzyl alcohol were found to have benzoic acid and benzyl alcohol respectively, as the dominant compound. It was furhter found that the benzoic acid and benzyl alcohol concentrations decreased over time. The sample comprising N-benzoyl-DL-leucine as the test compound was found to contain benzoic acid as the dominant compound, having a decreasing concentration over time. The sample comprising benzyl (s)-(−)-lactate as the test compound was found to contain benzyl alcohol as the dominant compound. Further, it was found that the concentration of benzyl alcohol increased over time. Finally, the sample comprising benzyl ethyl malonate as the test compound was found to contain benzyl alcohol as the dominant compound. Further, it was found that the concentration of benzyl alcohol increased over time.

In accordance with the present invention, the GC/MS analysis above show that the precursor compounds were broken down by the enzymes produced by S. aureus MN8 to produce the active species. It can further be seen that the precursor compounds were slowly broken down over time to allow for a long-lasting continous inhibition of exoprotein production by the active species.

Additional analysis of the 6 hour and 24 hour samples was conducted by liquid chromatography. In preparation for the chromatography, the samples were diluted 10 fold with water. The dilutions were then analyzed using an ion exclusion column to achieve the separation. Detection of the compounds was accomplished through UV absorbance at 230 nm.

In accordance with the present invention, the liquid chromatography analysis of the test compounds showed that the compounds were broken down into the active species, benzoic acid and benzyl alcohol, over a period of 24 hours. Specifically, the 6 hour and 24 hour samples containing 0.3% N-benzoyl-DL-leucine showed evidence of the compound's breakdown to benzoic acid. Additionally, the 6 hour and 24 hour samples containing benzyl (s)-(−)-lactate and benzyl ethyl malonate showed evidence of the compounds' breakdown to benzyl alcohol. As can further be seen, the 24 hour samples containing benzyl (s)-(−)-lactate and benzyl ethyl malonate showed evidence of an elevated level of benzyl alcohol compared to the 6 hour samples.

In view of the above, it will be seen that the several objects of the invention are achieved and other advantageous results obtained.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above without departing from the scope of the invention, it is intended that all matter contained in the above description and shown in the accompanying drawings shall be interpreted as illustrative and not in a limiting sense.

What is claimed is:

1. An absorbent article for inhibiting the production of exoprotein from Gram positive bacteria comprising an absorbent structure and an effective amount of a precursor compound having the general formula: 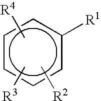
wherein R¹ is 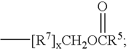
R⁷ is —OCH₂—; X is 0 or 1; R⁵ is a substituted or unsubstituted aromatic ring or a monovalent saturated or unsaturated, substituted or unsubstituted, branched or straight chain hydrocarbyl moiety that may or may not be substituted with hetero atoms; R², R³, and R⁴ are independently selected from the group consisting of H, OH, COOH, wherein upon hydrolysis the precursor compound is capable of producing an active species effective in inhibiting the production of exoprotein from Gram positive bacteria.

2. The absorbent article as set forth in claim 1 wherein R⁵ is a monovalent saturated, substituted or unsubstituted, branched or straight chain hydrocarbyl moiety that may or may not be substituted with hetero atoms having from 1 to 15 carbon atoms.

3. The absorbent article as set forth in claim 1 wherein R⁵ is a monovalent saturated, substituted or unsubstituted, branched or straight chain hydrocarbyl moiety that may or may not be substituted with hetero atoms having from 1 to 12 carbon atoms.

4. The absorbent article as set forth in claim 1 wherein R² is selected from the group consisting of H and OH, and R³ and R⁴ are independently H.

5. The absorbent article as set forth in claim 1 further comprising a surface-active agent selected from the group consisting of myreth-3-myristate, glycerol monolaurate, and laureth-4.

6. The absorbent article as set forth in claim 1 wherein the precursor compound is selected from the group consisting of benzyl(s)-(−)-lactate, benzyl ethyl malonate, benzyl-laurate, benzyl benzoate, benzyl paraben, benzyl salicylate, and phenoxyethyl paraben.

7. The absorbent article as set forth in claim 6 wherein the precursor compound is benzyl(s)-(−)-lactate.

8. The absorbent article as set forth in claim 7 further comprising a surface-active agent selected from the group consisting of myreth-3-myristate, glycerol monolaurate, and laureth-4.

9. The absorbent article as set forth in claim 6 wherein the precursor compound is benzyl ethyl malonate.

10. The absorbent article as set forth in claim 9 further comprising a surface-active agent selected from the group consisting of myreth-3-myristate, glycerol monolaurate, and laureth-4.

11. The absorbent article as set forth in claim 1 wherein the precursor compound is present in an amount of from about 0.15% (by weight of the absorbent structure) to about 2.0% (by weight of the absorbent structure).

12. The absorbent article as set forth in claim 1 wherein the precursor compound is present in an amount from about 0.17% (by weight of the absorbent structure) to about 1.7% (by weight of the absorbent structure).

13. The absorbent article as set forth in claim 1 wherein the precursor compound is microencapsulated in a shell material.

14. The absorbent article as set forth in claim 13 wherein the shell material comprises a material selected from the group consisting of cellulose-based polymeric materials, carbohydrate-based materials, and materials derived therefrom.

15. The absorbent article as set forth in claim 1 further comprising a pharmaceutically active material selected from the group consisting of selective antibacterials, antioxidants, anti-parasitic agents, antipruritics, astringents, local anaesthetics, and anti-inflammatory agents.

16. The absorbent article as set forth in claim 1 wherein the absorbent article is selected from the group consisting of a vaginal tampon, a sanitary napkin, a panty liner, an incontinent undergarment, a contraceptive sponge, a diaper, a wound dressing, a dental tampon, a medical tampon, a surgical tampon, and a nasal tampon.

17. An absorbent article for inhibiting the production of exoprotein from Gram positive bacteria comprising an absorbent structure and an effective amount of a precursor compound having the general formula: 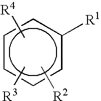
wherein R¹ is 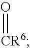
R⁶ is selected from the group consisting of an amino acid, a methyl ester of an amino acid, and an ethyl ester of an amino acid; R², R³, and R⁴ are independently selected from the group consisting of H, OH, COOH, wherein upon hydrolysis the precursor compound is capable of producing an active species effective in inhibiting the production of exoprotein from Gram positive bacteria.

18. The absorbent article as set forth in claim 17 wherein R⁶ is an amino acid.

19. The absorbent article as set forth in claim 18 wherein the amino acid is selected from the group consisting of valine, leucine, and cysteine.

20. The absorbent article as set forth in claim 17 further comprising a surface-active agent selected from the group consisting of myreth-3-myristate, glycerol monolaurate, and laureth-4.

21. The absorbent article as set forth in claim 17 wherein the precursor compound is selected from the group consisting of N-Benzoyl-DL-Valine, N-Benzoyl-DL-Leucine, and N-Benzoyl-DL-Cysteine.

22. The absorbent article as set forth in claim 21 further comprising a surface-active agent selected from the group consisting of myreth-3-myristate, glycerol monolaurate, and laureth-4.

23. The absorbent article as set forth in claim 17 wherein the precursor compound is present in an amount of from about 0.15% (by weight of the absorbent structure) to about 2.0% (by weight of the absorbent structure).

24. The absorbent article as set forth in claim 17 wherein the precursor compound is present in an amount from about 0.17% (by weight of the absorbent structure) to about 1.7% (by weight of the absorbent structure).

25. The absorbent article as set forth in claim 17 wherein the precursor compound is microencapsulated in a shell material.

26. The absorbent article as set forth in claim 25 wherein the shell material comprises a material selected from the group consisting of cellulose-based polymeric materials, carbohydrate-based materials, and materials derived therefrom.

27. The absorbent article as set forth in claim 17 further comprising a pharmaceutically active material selected from the group consisting of selective antibacterials, antioxidants, anti-parasitic agents, antipruritics, astringents, local anaesthetics, and anti-inflammatory agents.

28. The absorbent article as set forth in claim 17 wherein the absorbent article is selected from the group consisting of a vaginal tampon, a sanitary napkin, a panty liner, an incontinent undergarment, a contraceptive sponge, a diaper, a wound dressing, a dental tampon, a medical tampon, a surgical tampon, and a nasal tampon.

29. A vaginal tampon for inhibiting the production of exoprotein from Gram positive bacteria comprising an absorbent tampon material and an effective amount of a precursor compound having the general formula: 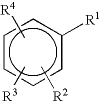
wherein R¹ is 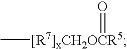
R⁷ is —OCH₂—; X is 0 or 1; R⁵ is a substituted or unsubstituted aromatic ring or a monovalent saturated or unsaturated, substituted or unsubstituted, branched or straight chain hydrocarbyl moiety that may or may not be substituted with hetero atoms; R², R³, and R⁴ are independently selected from the group consisting of H, OH, COOH, wherein upon hydrolysis the precursor compound is capable of producing an active species effective in inhibiting the production of exoprotein from Gram positive bacteria.

30. The vaginal tampon as set forth in claim 29 wherein R⁵ is a monovalent saturated, substituted or unsubstituted, branched or straight chain hydrocarbyl moiety that may or may not be substituted with hetero atoms having from 1 to 15 carbon atoms.

31. The vaginal tampon as set forth in claim 29 wherein R⁵ is a monovalent saturated, substituted or unsubstituted, branched or straight chain hydrocarbyl moiety that may or may not be substituted with hetero atoms having from 1 to 12 carbon atoms.

32. The vaginal tampon as set forth in claim 29 wherein R² is selected from the group consisting of H and OH, and R³ and R⁴ are independently H.

33. The vaginal tampon as set forth in claim 29 further comprising a surface-active agent selected from the group consisting of myreth-3-myristate, glycerol monolaurate, and laureth-4.

34. The vaginal tampon as set forth in claim 29 wherein the precursor compound is selected from the group consisting of benzyl(s)-(−)-lactate, benzyl ethyl malonate, benzyl-laurate, benzyl benzoate, benzyl paraben, benzyl salicylate, and phenoxyethyl paraben.

35. The vaginal tampon as set forth in claim 34 wherein the precursor compound is benzyl(s)-(−)-lactate.

36. The vaginal tampon as set forth in claim 35 further comprising a surface-active agent selected from the group consisting of myreth-3-myristate, glycerol monolaurate, and laureth-4.

37. The vaginal tampon as set forth in claim 36 wherein the precursor compound is benzyl ethyl malonate.

38. The vaginal tampon as set forth in claim 37 further comprising a surface-active agent selected from the group consisting of myreth-3-myristate, glycerol monolaurate, and laureth-4.

39. The vaginal tampon as set forth in claim 29 wherein the precursor compound is present in an amount of at least about 0.15% (by weight of the absorbent tampon material) to about 2.0% (by weight of the absorbent tampon material).

40. The vaginal tampon as set forth in claim 29 wherein the precursor compound is present in an amount from about 0.17% (by weight of the absorbent tampon material) to about 1.7% (by weight of the absorbent tampon material).

41. The vaginal tampon as set forth in claim 29 wherein the precursor compound is microencapsulated in a shell material.

42. The vaginal tampon as set forth in claim 41 wherein the shell material comprises a material selected from the group consisting of cellulose-based polymeric materials, carbohydrate-based materials, and materials derived therefrom.

43. The vaginal tampon as set forth in claim 29 further comprising a pharmaceutically active material selected from the group consisting of selective antibacterials, antioxidants, anti-parasitic agents, antipruritics, astringents, local anaesthetics, and anti-inflammatory agents.

44. A vaginal tampon for inhibiting the production of exoprotein from Gram positive bacteria comprising an absorbent tampon material and an effective amount of an precursor compound having the general formula: 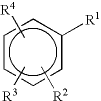
wherein R¹ is 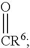
R⁶ is selected from the group consisting of an amino acid, a methyl ester of an amino acid, and an ethyl ester of an amino acid; R², R³, and R⁴ are independently selected from the group consisting of H, OH, COOH, wherein upon hydrolysis the precursor compound is capable of producing an active species effective in inhibiting the production of exoprotein from Gram positive bacteria.

45. The vaginal tampon as set forth in claim 44 wherein R⁶ is an amino acid.

46. The vaginal tampon as set forth in claim 45 wherein the amino acid is selected from the group consisting of valine, leucine, and cysteine.

47. The vaginal tampon as set forth in claim 44 further comprising a surface-active agent selected from the group consisting of myreth-3-myristate, glycerol monolaurate, and laureth-4.

48. The vaginal tampon as set forth in claim 44 wherein the precursor compound is selected from the group consisting of N-Benzoyl-DL-Valine, N-Benzoyl-DL-Leucine, and N-Benzoyl-DL-Cysteine.

49. The vaginal tampon as set forth in claim 48 further comprising a surface-active agent selected from the group consisting of myreth-3-myristate, glycerol monolaurate, and laureth-4.

50. The vaginal tampon as set forth in claim 44 wherein the precursor compound is present in an amount of from about 0.15% (by weight of the absorbent tampon material) to about 2.0% (by weight of the absorbent tampon material).

51. The vaginal tampon as set forth in claim 44 wherein the precursor compound is present in an amount from about 0.17% (by weight of the absorbent tampon material) to about 1.7% (by weight of the absorbent tampon material).

52. The vaginal tampon as set forth in claim 44 wherein the precursor compound is microencapsulated in a shell material.

53. The vaginal tampon as set forth in claim 52 wherein the shell material comprises a material selected from the group consisting of cellulose-based polymeric materials, carbohydrate-based materials, and materials derived therefrom.

54. The vaginal tampon as set forth in claim 44 further comprising a pharmaceutically active material selected from the group consisting of selective antibacterials, antioxidants, anti-parasitic agents, antipruritics, astringents, local anaesthetics, and anti-inflammatory agents.

55. A vaginal tampon for inhibiting the production of exoprotein from Gram positive bacteria comprising an absorbent tampon material and a cover material, wherein the cover material comprises an effective amount of a precursor compound having the general formula: 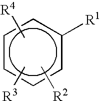
wherein R¹ is 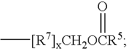
R⁷ is —OCH₂—; X is 0 or 1; R⁵ is a substituted or unsubstituted aromatic ring or a monovalent saturated or unsaturated, substituted or unsubstituted, branched or straight chain hydrocarbyl moiety that may or may not be substituted with hetero atoms; R², R³, and R⁴ are independently selected from the group consisting of H, OH, COOH, wherein upon hydrolysis the precursor compound is capable of producing an active species effective in inhibiting the production of exoprotein from Gram positive bacteria.

56. The vaginal tampon as set forth in claim 55 wherein R⁵ is a monovalent saturated, substituted or unsubstituted, branched or straight chain hydrocarbyl moiety that may or may not be substituted with hetero atoms having from 1 to 15 carbon atoms.

57. The vaginal tampon as set forth in claim 55 wherein R⁵ is a monovalent saturated, substituted or unsubstituted, branched or straight chain hydrocarbyl moiety that may or may not be substituted with hetero atoms having from 1 to 12 carbon atoms.

58. The vaginal tampon as set forth in claim 55 wherein R² is selected from the group consisting of H and OH, and R³ and R⁴ are independently H.

59. The vaginal tampon as set forth in claim 55 further comprising a surface-active agent selected from the group consisting of myreth-3-myristate, glycerol monolaurate, and laureth-4.

60. The vaginal tampon as set forth in claim 55 wherein the precursor compound is selected from the group consisting of benzyl(s)-(−)-lactate, benzyl ethyl malonate, benzyl-laurate, benzyl benzoate, benzyl paraben, benzyl salicylate, and phenoxyethyl paraben.

61. The vaginal tampon as set forth in claim 60 wherein the precursor compound is benzyl(s)-(−)-lactate.

62. The vaginal tampon as set forth in claim 61 further comprising a surface-active agent selected from the group consisting of myreth-3-myristate, glycerol monolaurate, and laureth-4.

63. The vaginal tampon as set forth in claim 60 wherein the precursor compound is benzyl ethyl malonate.

64. The vaginal tampon as set forth in claim 63 further comprising a surface-active agent selected from the group consisting of myreth-3-myristate, glycerol monolaurate, and laureth-4.

65. The vaginal tampon as set forth in claim 55 wherein the precursor compound is present in an amount of at least about 2.6% (by weight of the cover material) to about 35% (by weight of the cover material).

66. The vaginal tampon as set forth in claim 55 wherein the precursor compound is present in an amount from about 2.95% (by weight of the cover material) to about 29.5% (by weight of the cover material).

67. The vaginal tampon as set forth in claim 55 wherein the precursor compound is microencapsulated in a shell material.

68. The vaginal tampon as set forth in claim 67 wherein the shell material comprises a material selected from the group consisting of cellulose-based polymeric materials, carbohydrate-based materials, and materials derived therefrom.

69. The vaginal tampon as set forth in claim 55 further comprising a pharmaceutically active material selected from the group consisting of selective antibacterials, antioxidants, anti-parasitic agents, antipruritics, astringents, local anaesthetics, and anti-inflammatory agents.

70. A vaginal tampon for inhibiting the production of exoprotein from Gram positive bacteria comprising an absorbent tampon material and a cover material, wherein the cover material comprises an effective amount of an precursor compound having the general formula: 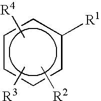
wherein R¹ is 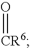
R⁶ is selected from the group consisting of an amino acid, a methyl ester of an amino acid, and an ethyl ester of an amino acid; R², R³, and R⁴ are independently selected from the group consisting of H, OH, COOH, wherein upon hydrolysis the precursor compound is capable of producing an active species effective in inhibiting the production of exoprotein from Gram positive bacteria.

71. The vaginal tampon as set forth in claim 70 wherein R⁶ is an amino acid.

72. The vaginal tampon as set forth in claim 71 wherein the amino acid is selected from the group consisting of valine, leucine, and cysteine.

73. The vaginal tampon as set forth in claim 70 further comprising a surface-active agent selected from the group consisting of myreth-3-myristate, glycerol monolaurate, and laureth-4.

74. The vaginal tampon as set forth in claim 70 wherein the precursor compound is selected from the group consisting of N-Benzoyl-DL-Valine, N-Benzoyl-DL-Leucine, and N-Benzoyl-DL-Cysteine.

75. The vaginal tampon as set forth in claim 74 further comprising a surface-active agent selected from the group consisting of myreth-3-myristate, glycerol monolaurate, and laureth-4.

76. The vaginal tampon as set forth in claim 70 wherein the precursor compound is present in an amount of from about 2.6% (by weight of the cover material) to about 35% (by weight of the cover material).

77. The vaginal tampon as set forth in claim 70 wherein the precursor compound is present in an amount from about 2.95% (by weight of the cover material) to about 29.5% (by weight of the cover material).

78. The vaginal tampon as set forth in claim 70 wherein the precursor compound is microencapsulated in a shell material.

79. The vaginal tampon as set forth in claim 78 wherein the shell material comprises a material selected from the group consisting of cellulose-based polymeric materials, carbohydrate-based materials, and materials derived therefrom.

80. The vaginal tampon as set forth in claim 70 further comprising a pharmaceutically active material selected from the group consisting of selective antibacterials, antioxidants, anti-parasitic agents, antipruritics, astringents, local anaesthetics, and anti-inflammatory agents.
